(19) United States
(12) Patent Application Publication
(10) Pub. No.: US 2006/0134057 A1
(43) Pub. Date: Jun. 22, 2006

(54) Cosmetic composition comprising a polyol ether and an ester

(76) Inventors: Audrey Ricard, Paris (FR); Carole Le Merrer, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/274,345

(22) Filed: Nov. 16, 2005

Related U.S. Application Data
(60) Provisional application No. 60/629,326, filed on Nov. 19, 2004.

(30) Foreign Application Priority Data
Nov. 16, 2004 (FR) 04 52636

(51) Int. Cl. A61K 8/39; A61K 8/37

(52) U.S. Cl. 424/70.31

(57) ABSTRACT

Disclosed herein is a composition comprising, in at least one physiologically acceptable medium, at least one fatty phase comprising at least one polyol ether and at least one neoacid ester. The polyol ether may be, for example, a pentaerythritol ether. This composition may, for instance, constitute a stick and may be used as a care and/or make-up product for keratin materials, such as the skin, the lips and/or the superficial body growths. Cosmetic methods are also disclosed.

This application claims benefit of U.S. Provisional Application No. 60/629,326, filed Nov. 19, 2004, the contents of which are incorporated herein by reference. This application also claims benefit of priority under 35 U.S.C. § 119 to French Patent Application No. 04 52636, filed Nov. 16, 2004, the contents of which are also incorporated by reference.

The present disclosure relates to a composition comprising a fatty phase comprising at least one particular polyol ether. The composition may comprise at least one hydrocarbon ester, for example a neoacid ester. This composition may be used as make-up and/or care composition for keratin materials such as the skin, the lips and the superficial body growths.

European Patent Application No. EP 1 306 074 describes a solid composition comprising a polyol ether, an oil and a wax of reversed polarity. This document describes how this combination makes it possible to obtain a solid composition which does not melt at a temperature of less than or equal to 37° C., while spreading easily on the skin to give a good deposit.

Disclosed herein are cosmetic compositions which may be in the form of a stick or a paste. These compositions may, for example, have better retention on the keratin materials than the prior art compositions comprising a polyol ether, while also having a good level of comfort.

More specifically, the present disclosure relates to cosmetic compositions comprising, in a physiologically acceptable medium, at least one fatty phase comprising at least one polyol ether chosen from polyalkylene glycol pentaerythritol ethers, and at least one hydrocarbon ester.

In at least one embodiment, the compositions according to the present disclosure may comprise a single fatty phase, which can be, for instance, a continuous phase.

The compositions according to the present disclosure may be anhydrous compositions, and may then comprise up to 10% by weight of hydrophilic phase relative to the total weight of the composition, such as 1 to 5% by weight of hydrophilic phase and, for example, 1 to 2% of hydrophilic phase relative to the total weight of the composition, the hydrophilic phase comprising water alone or a mixture of water and hydrophilic and water-soluble additives such as polyols, gelling agents and/or active agents. If this hydrophilic phase is present, it can, in at least one embodiment, be dispersed in the fatty phase which forms a continuous phase.

According to one embodiment of the present disclosure, the composition is anhydrous, that is to say that it comprises only the fatty phase, or is practically anhydrous, that is to say that it comprises less than 5% by weight of water and/or hydrophilic or water-soluble additives.

In one embodiment, disclosed herein is a composition comprising, in a physiologically acceptable medium, at least one fatty phase comprising at least one polyol ether chosen from polyalkylene glycol pentaerythritol ethers, and at least one ester resulting from the reaction of an alcohol with a carboxylic acid of the following formula (I): 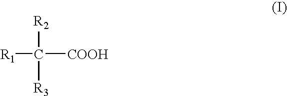
wherein R₁, R₂ and R₃, which may be identical or different, are radicals chosen from alkyl, aryl and aralkyl radicals, and combinations thereof, wherein the carboxylic acid of formula (I) is referred to below as a “neoacid”.

In another embodiment, disclosed herein is a composition comprising, in a physiologically acceptable medium, at least one fatty phase comprising at least one polyol ether, at least one hydrocarbon ester and a non-volatile oil different from the hydrocarbon ester.

According to yet another embodiment, disclosed herein is a composition comprising, in a physiologically acceptable medium, at least one fatty phase comprising at least one polyol ether and at least two hydrocarbon esters. One of the two esters may be a neoacid ester of formula (I) described above. The other of the two esters may be a monoester comprising, for example, from 18 to 26 carbon atoms. For instance, the neoacid ester and the monoester may be present in the composition in a ratio by mass ranging from 2/1 to 1/5, such as ranging from 1/1 to 1/5, and further, for example, ranging from 1/1 to 1/3, such as a ratio of 1/2.

According to this embodiment of the disclosure, the acid ester of formula (I) may be present in the composition at a concentration ranging from 5 to 35%, such as, for example ranging from 10 to 15%. The other hydrocarbon ester may be present at a concentration of ranging from 10 to 50% by weight, such as ranging from 15 to 30% by weight relative to the total weight of the composition.

According to another embodiment, the present disclosure relates to a composition comprising, in a physiologically acceptable medium, at least one fatty phase comprising at least one polyol ether, a pasty compound other than the polyol ether, and at least one hydrocarbon ester.

Polyol Ethers

In the compositions according to the present disclosure, the at least one polyol ether may be chosen from, for instance, polyalkylene glycol pentaerythritol ethers, sugar fatty alcohol ethers and mixtures thereof.

The polyalkylene glycol pentaerythritol ethers may comprise, for example, from 1 to 450 oxyalkylenated units, for instance from 1 to 200 oxyalkylenated units, such as from 1 to 100 oxyalkylenated units or from 1 to 50 oxyalkylenated units. In at least one embodiment, these ethers may be chosen from polyethylene glycol pentaerythritol ethers comprising from 1 to 450 oxyethylenated units, for example, from 1 to 200 oxyethylenated units, further, for example from 1 to 100 oxyethylenated units or from 1 to 50 oxyethylenated units; polypropylene glycol pentaerythritol ethers comprising from 1 to 450 oxypropylenated units, for example, from 1 to 200 oxypropylenated units, such as from 1 to 100 oxypropylenated units or from 1 to 50 oxypropylenated units; and mixtures thereof.

In one embodiment of the present disclosure, the at least one polyol ether may be chosen from the polyethylene glycol pentaerythrityl ether comprising 5 oxyethylenated units (5 EO) (CTFA name: PEG-5 Pentaerythrityl Ether), the polypropylene glycol pentaerythritol ether comprising 5 oxypropylenated units (5 PO) (CTFA name: PPG-5 Pentaerythrityl Ether), and mixtures thereof. For example, the PEG-5 Pentaerythrityl Ether, PPG-5 Pentaerythrityl Ether and soya bean oil mixture marketed under the name “Lanolide” by the company Vevy may be used; in this mixture the constituents are in a 46/46/8 weight ratio: 46% of PEG-5 Pentaerythrityl Ether, 46% of PPG-5 Pentaerythrityl Ether and 85% of soya bean oil.

The sugar fatty alcohol ethers may be chosen from the group comprising C₈-C₂₂ fatty alcohol ethers or mixtures of C₈-C₂₂ fatty alcohol ethers of glucose, maltose, sucrose or fructose; C₁₄-C₂₂ fatty alcohol ethers or mixtures of C₁₄-C₂₂ fatty alcohol ethers of methylglucose, and mixtures thereof.

C₈-C₂₂ or C₁₄-C₂₂ fatty alcohols forming the fatty unit of the sugar ethers may comprise a saturated or unsaturated linear alkyl chain comprising from 8 to 22 or from 14 to 22 carbon atoms respectively. The fatty unit of the ethers obtained from the fatty alcohol may be chosen, for example, from decyl, cetyl, behenyl, arachidyl, stearyl, palmityl, myristyl, lauryl, capryl and hexadecanyl units, and mixtures thereof such as cetearyl (mixture of cetyl and stearyl).

By way of non-limiting examples of sugar fatty alcohol ethers, there may be mentioned alkyl polyglucosides (APG) such as decyl glucoside and lauryl glucoside marketed for example by the company Henkel under the respective names PLANTAREN 2000 and PLANTAREN 1200, and cetearyl glucoside optionally as a mixture with cetostearyl alcohol, the mixture being marketed for example under the name MONTANOV 68 by the company Seppic (mixture wherein the constituents are in a 12/46/42 ratio: 12% of cetearyl glucoside, 46% of cetyl alcohol and 42% of stearyl alcohol). As cetearyl glucoside, there may also be mentioned the products marketed under the name TEGOCARE CG90 by the company Goldschmidt and under the name EMULGADE KE3302 by the company Henkel. As APG, there may also be mentioned arachidyl glucoside, for example in the form of the mixture of arachidyl and behenyl alcohols and arachidyl glucoside, marketed under the name MONTANOV 202 by the company Seppic. There may also be mentioned the branched or unsaturated chain alkyl polyglucosides such as isostearyl glucoside optionally as a mixture with isostearyl alcohol, marketed for example under the name MONTANOV WO13 by the company Seppic, and oleyl glucoside optionally as a mixture with oleyl alcohol, marketed by the company Seppic. It is also possible to use a mixture of these alkyl polyglucosides. According to one embodiment of the present disclosure, the mixture of the alkyl polyglucoside as defined above with the corresponding fatty alcohol may be in the form of a self-emulsifiable composition as described for example in International Patent Publication No. WO-A-92/06778.

The amount of the at least one polyol ether used in the presently disclosed compositions can vary widely, and it may range for example from 0.5 to 40% by weight, such as from 1 to 30% by weight, and further, for example from 50 to 25% by weight relative to the total weight of the composition.

Hydrocarbon Esters

The expression “hydrocarbon ester” as used herein is understood to mean a compound comprising at least one ester functional group COO.

The word “ester”, as used herein, is understood to mean a monoester, a diester, a triester or more generally a polyester. The ester of the compositions according to the present disclosure may result from a total or partial esterification (in the latter case, the ester comprises at least one free —OH functional group).

As used herein, the expression “at least one” ester is understood to mean one or more esters.

As used herein, the expression “physiologically acceptable” is understood to mean non-toxic and capable of being applied to the skin, the superficial body growths or the lips of human beings.

The physiologically acceptable medium for the presently disclosed compositions is, in at least one embodiment, cosmetically acceptable, i.e., having a pleasant taste, feel, appearance and/or smell, and/or capable of being applied for several days and/or for several months.

According to the present disclosure, the hydrocarbon ester can be, for example, an oil, i.e., a fatty substance which is liquid at atmospheric pressure and at a temperature of 23° C.

The hydrocarbon ester may be linear, branched or cyclic, saturated or unsaturated.

The hydrocarbon ester can be, for example, a non-volatile oil. The expression “non-volatile oil” as used herein is understood to mean an oil (or a non-aqueous medium) having a vapor pressure of less than 0.13 Pa.

For instance, the hydrocarbon ester may correspond to the formula RCOOR′ wherein RCOO is chosen from a carboxylic acid residue comprising from 2 to 30 carbon atoms, and R′ is chosen from a hydrocarbon chain comprising from 1 to 30 carbon atoms.

RCOO is, for example, the residue of an aliphatic carboxylic acid comprising 2 to 30 carbon atoms, such as from 4 to 26 carbon atoms, and for example from 4 to 22 carbon atoms.

In at least one embodiment, the radical R is chosen from an alkyl radical and an alkenyl radical. As used herein, the expression “alkyl radical” is understood to mean a linear or branched, and saturated aliphatic radical comprising carbon and hydrogen. The expression “alkenyl radical,” as used herein, is understood to mean a linear or branched, and unsaturated aliphatic radical comprising carbon and hydrogen, i.e., comprising at least one carbon-carbon double bond, for example, from one to three double bonds. In at least one embodiment, the alkenyl radical comprises one carbon-carbon double bond.

RCOO may be chosen from the residue of a fatty acid; e.g., an acid obtained by hydrolysis of fatty substances of plant or animal origin.

The radical R′O is chosen from the residue of an alcohol, for example, a linear or branched, saturated or unsaturated aliphatic alcohol. The radical R′ can be, for instance, an alkyl radical or an alkenyl radical, independently of the choice of the radical R, alkyl and alkenyl being as defined above.

R′O may be chosen from the residue of a fatty alcohol, i.e., an alcohol obtained by hydrogenation of a fatty acid as defined above.

In at least one embodiment, R′O is the residue of an aliphatic alcohol comprising 2 to 30 carbon atoms, such as from 4 to 26 carbon atoms, further, for example from 4 to 22 carbon atoms.

R and R′ are chosen independently of each other. In at least one embodiment, they are chosen so that they are both saturated and branched, or both linear and monounsaturated.

The at least one ester may comprise up to 60 carbon atoms, such as from 10 to 45, and further, for example from 18 to 40 carbon atoms.

According to one embodiment of the present disclosure, the hydrocarbon ester is a branched and saturated monoester. The ester can be, for example, a monoester of a branched and saturated aliphatic carboxylic acid and of a branched and saturated aliphatic alcohol.

According to another embodiment of the present disclosure, the hydrocarbon ester can be a monoester of a monounsaturated (comprising one carbon-carbon double bond) and linear fatty acid, and of a monounsaturated and linear fatty alcohol.

Thus, the esters may be chosen from a non-limiting list comprising
isononanoic acid esters such as isononyl isononanoate, octyl isononanoate, isodecyl isononanoate, isotridecyl isononanoate, isostearyl isononanoate,
isopropyl alcohol esters such as isopropyl myristate, isopropyl palmitate, isopropyl stearate or isostearate,
fatty acid esters such as purcellin oil, ethyl palmitate, and octyl stearate,
hydroxylated esters such as isostearyl lactate, octyl hydroxystearate, diisopropyl adipate,
fatty alcohol heptanoates, octanoates or decanoates such as cetyl octanoate and tridecyl octanoate,
and mixtures thereof.

The hydrocarbon ester may also be chosen from neoacid esters. The expression “neoacid ester” as used herein is understood to mean an ester resulting from the reaction of an alcohol with a carboxylic acid of the following formula (I): 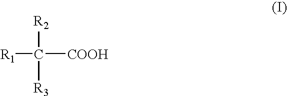
wherein R₁, R₂ and R₃ are radicals, which may be identical or different, chosen from optionally functionalized alkyl, aryl and aralkyl radicals, and combinations thereof.

The neocarboxylic acid esters may be chosen from neopentanoic acid esters such as isodecyl neopentanoate, isotridecyl neopentanoate, isostearyl neopentanoate and 2-octyidodecyl neopentanoate.

The carbon at the alpha-position of the acid functional group of the carboxylic acid of formula (I) is trisubstituted with radicals, which may be identical or different, chosen from alkyl, aryl and aralkyl radicals, and combinations thereof. This carbon atom can be, therefore, directly linked to 4 carbon atoms and is called “neo” carbon. An acid comprising such a structure is called “neo” acid and will be thus designated in the remainder of the text.

The expression “alkyl radical,” as used herein, is understood to mean a branched or unbranched, and saturated aliphatic hydrocarbon chain. In one embodiment, an alkyl radical is understood to comprise, for instance, from 1 to 28 carbon atoms.

As used herein, the word “branched” is understood to mean at least one hydrocarbon pendant chain comprising at least one carbon atom.

The expression “aryl radical,” as used herein, is understood to mean a radical derived from an aromatic cyclic compound by removal of a hydrogen atom such as, for example, phenyl or tolyl radicals.

The expression “aralkyl radical,” as used herein, is understood to mean an alkyl chain substituted with an aryl radical, for example of the R′—C₆H₅ type, R′ being a C₁-C₅ alkyl, such as, for example, benzyl or phenethyl radicals.

These alkyl, aryl or aralkyl radicals may be functionalized, which is understood herein to mean that they may comprise in their structure a heteroatom, i.e., an atom different from the carbon atom (such as an oxygen, nitrogen, sulphur or fluorine atom) or a functional group such as a COOH, OH, NHR or COOR group.

The “neo” carboxylic acid may be a monoacid or a polyacid, with at least one of the radicals R₁, R₂ or R₃ comprising a functional group —COOH; for example, the carboxylic acid can be a monoacid The radicals R₁, R₂ and R₃, which may be identical or different, can be chosen from non-functionalized alkyl, aryl or aralkyl radicals.

For instance, the R₁, R₂ and R₃ radicals, which may be identical or different, can be chosen from saturated alkyl radicals, such as from saturated C₁-C₁₅, for example C₁-C₆, alkyl radicals such as for example methyl, ethyl, propyl, isopropyl, pentyl or hexyl radical.

In at least one embodiment, the “neo” carboxylic acid comprises a total number of carbon atoms ranging from 5 to 30, such as, for example, from 5 to 15.

In at least one embodiment, a “neo” carboxylic acid chosen from the neopentanoic acid of formula CH₃—C(CH₃)₂—COOH, the neoheptanoic acid of formula C₃H₇—C(CH₃)₂—COOH, the neodecanoic acid of formula C₆H₁₃—C(CH₃)₂—COOH and mixtures thereof, may be used. For example, in one embodiment, the “neo” carboxylic acid may be neopentanoic acid.

The ester of the composition according to the present disclosure results from the reaction of a “neo” carboxylic acid as described above, with an alcohol which may be a monoalcohol or a polyol.

According to one embodiment of the present disclosure, the alcohol can be a compound of formula R′OH wherein R′O is defined above. In one embodiment, the alcohol comprises a number of carbon atoms ranging from 2 to 20. The alcohol may be, for example, a fatty alcohol.

The alcohol may be chosen from saturated, linear and branched monoalcohols comprising from 16 to 20 carbon atoms.

The alcohol may also be a polyol chosen from “neo” polyols, that is to say polyols which comprise a carbon atom, at the alpha position of one of the alcohol functional groups, which is trisubstituted with radicals independently chosen from alkyl, aryl and aralkyl radicals, and combinations thereof. This carbon atom directly linked to 4 other carbon atoms is therefore a “neo” carbon as defined above.

As “neo” polyol, the neopentyl glycol of formula HOCH₂—C(CH₃)₂—CH₂—OH, the trimethylolpropane of formula HOCH₂—C(C₂H₅)(CH₂—OH)₂, the pentaerythritol of formula HOCH₂—C(CH₂OH)₂—CH₂—OH and mixtures thereof, may be used. In at least one embodiment, the polyol is neopentyl glycol.

The hydrocarbon ester of the compositions according to the present disclosure may be present in an amount ranging from 0.1 to 99.9%, for example from 1 to 99%, and further, for example from 5 to 90% of the total weight of the composition. For example, the hydrocarbon ester may be present in an amount ranging from 5 to 60% of the total weight of the composition.

Oils

The fatty phase of the compositions according to the present disclosure may comprise at least one oil, in addition to the hydrocarbon ester described above. As used herein, the expression “oil” is understood to mean any physiologically acceptable non-aqueous medium which is liquid at room temperature (25° C.) and atmospheric pressure (760 mmHg).

The quantity of oil(s) may range for example from 20 to 80% by weight, and such as, for example from 30 to 70% by weight relative to the total weight of the composition.

These oils may be hydrocarbon and/or silicone and/or fluorinated oils. They may be of animal, plant, mineral or synthetic origin. The expression “hydrocarbon oil,” as used herein, is understood to mean any oil predominantly comprising carbon and hydrogen atoms, and optionally ester, ether, fluorinated, carboxylic acid and/or alcohol groups. In addition, the oils used may be volatile and/or non-volatile. As used herein, the expression “volatile oil” is understood to mean an oil capable of evaporating at room temperature from a support onto which it is applied, in other words an oil having a vapor pressure which is measurable at 25° C. and 1 atmosphere, for example greater than 0 Pa, such as ranging from 10⁻³ to 300 mmHg (0.13 Pa to 40 000 Pa). Non-limiting examples of volatile oils may be volatile silicone oils such as cyclic or linear volatile silicones. There may also be mentioned volatile hydrocarbon oils such as isoparaffins and fluorinated volatile oils.

Among the oils which may be used in the composition of the disclosure, some are polar and others are apolar (that is to say non-polar).

In their chemical structure, the polar oils comprise at least one non-ionic polar group, and may comprise, for example, at least two non-ionic or ionic polar groups such as the following groups:
COOH;
OH which is mono or disubstituted (primary or secondary);
PO₄;
NHF; NR₁R₂, R₁ and R₂ optionally forming a ring and chosen from a linear or branched C₁ to C₂₀ alkyl and alkoxy radical, or 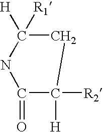
   where R₁′ and R₂′ may be chosen from hydrogen, linear or branched C₁ to C₂₀ alkyl chains, and linear or branched C₁ to C₂₀ alkoxy chains.

The polarity may be described by the Hansen solubility parameter δₐ. This parameter characterizes, for a given constituent, the energy corresponding to the polar interactions (δₚ) and the hydrogen-bond-type interactions (δₕ) existing between the molecules of this constituent.

$${δ}_{a} = \sqrt{{δ}_{p}^{2} + {δ}_{h}^{2}}$$

According to the present disclosure, the apolar oils have a δₐ value equal to 0. In at least one embodiment, the apolar oils may be chosen from:
silicone oils, such as linear or cyclic, volatile or non-volatile polydimethylsiloxanes (PDMS) which are liquid at room temperature; phenylated silicones such as phenyltrimethicones, phenlydimethicones, phenyltrimethylsiloxydiphenylsiloxanes, diphenyidimethicones, diphenylmethyldiphenyltrisiloxanes, 2-phenylethyl trimethylsiloxysilicates;
linear or branched hydrocarbons of synthetic or inorganic origin such as volatile or non-volatile paraffin oils and derivatives thereof; liquid paraffin; liquid lanolin; polydecenes; hydrogenated polyisobutene such as Parleam® oil; squalane; hydrogenated isoparaffin; isohexadecane; isododecane;
and mixtures thereof.

The polar oils have a δₐ value different than 0, i.e., greater than 0. In at least one embodiment, the polar oils used in the composition of the disclosure may be chosen from:
oils of plant origin, hydrocarbon oils with a high content of triglycerides comprising fatty acid esters of glycerol wherein the fatty acids may have varying chain lengths from C₄ to C₂₀, it being possible for the latter to be linear or branched, saturated or unsaturated. As oils of plant origin, there may be mentioned, for example, jojoba, wheat germ, maize, sunflower, shea butter, castor, sweet almond, macadamia, apricot, soya bean, cottonseed, lucerne, poppyseed, pumpkinseed, sesame, gourd, rapeseed, avocado, hazelnut, grapeseed, blackcurrant seed, evening primrose, millet, barley, quinoa, olive, rye, safflower, candlenut, passionflower, rose-musk and coriander oils; or alternatively caprylic/capric acid triglycerides such as those sold by the company Stearineries Dubois or those sold under the names MIGLYOL 810, 812 and 818 by the company Dynamit Nobel;
synthetic oils or synthetic esters of formula R₅COOR₆ wherein R₅ is chosen from the residue of a linear or branched fatty acid comprising from 1 to 40 carbon atoms and R₆ is chosen from a hydrocarbon chain, for example a branched hydrocarbon chain, comprising from 1 to 40 carbon atoms, provided that R₅+R₆ is ≧10, such as for example Purcellin oil (cetostearyl octanoate), isononyl isononanoate, C₁₂ to C₁₅ alcohol benzoate, isoprypyl myristate, 2-ethylhexyle palmitate, isostearyl isostearate, isoprypyl isostearate, alcohol or polyalcohol octanoates, decanoates or ricinoleates; hydroxylated esters such as isostearyl lactate, diisostearyl malate; C₁₂-C₁₅ alkyl benzoate; and pentaerythritol esters;
synthetic ethers having from 10 to 40 carbon atoms;
C₈ to C₂₆ fatty alcohols such as oleyl alcohol, isostearyl alcohol and octyldodecanol;

mixtures thereof.

Pasty Substances

The compositions according to the present disclosure may additionally comprise at least one pasty compound different from the polyol ether described above.

The expression “pasty”, for the purposes of the present disclosure, is understood to mean a lipophilic fatty compound with a reversible solid/liquid change of state having, in the solid state, an anisotropic crystalline organization, and having, at the temperature of 23° C., a liquid fraction and a solid fraction. The expression “pasty” is also understood to mean polyvinyl laurate.

The pasty compound, for the purposes of the disclosure, may have a hardness, at 20° C., ranging from 0.001 to 0.5 MPa, for example, from 0.002 to 0.4 MPa.

The hardness is measured by a method of penetration of a probe into a sample of compound with the aid of a texture analyzer (for example the TA-XT2i from Rheo) equipped with a stainless steel spindle 2 mm in diameter. The measurement of hardness can be performed at 20° C. at the center of 5 samples. The spindle is introduced into each sample at a pre-speed of 1 mm/s and then at a measuring speed of 0.1 mm/s, the penetration depth being 0.3 mm. The hardness value measured is that of the maximum peak.

The pasty compound may, at a temperature of 23° C., be in the form of a liquid fraction and of a solid fraction. In other words, the onset of melting temperature of the pasty compound may be less than 23° C. The liquid fraction of the pasty compound, measured at 23° C., may comprise from 9 to 97% by weight of the compound, such as from 15 to 85% by weight, for example from 40 to 85% by weight.

The liquid fraction by weight of the pasty compound at 23° C. is equal to the ratio of the heat of fusion consumed at 23° C. to the heat of fusion of the pasty compound.

The heat of fusion of the pasty compound is the heat consumed by the compound to change from the solid state to the liquid state. The pasty compound is said to be in the solid state when all of its mass is in the solid crystalline form. The pasty compound is said to be in the liquid state when all of its mass is in the liquid form.

The heat of fusion of the pasty compound is equal to the area under the curve of the thermogram obtained with the aid of a differential scanning calorimeter (D.S.C.), such as the calorimeter sold under the name MDSC 2920 by the company TA Instrument, with a temperature rise of 5 or 10° C. per minute, according to the ISO 11357-3:1999 standard. The heat of fusion of the pasty compound is the amount of energy required to make the compound change from the solid state to the liquid state. It is expressed in J/g.

The heat of fusion consumed at 23° C. is the amount of energy absorbed by the sample to change from the solid state to the state that it has at 23° C., comprising a liquid fraction and a solid fraction.

The liquid fraction of the pasty compound measured at 32° C., comprises from 30 to 100% by weight of the compound, such as, for example from 80 to 100%, and further, for example 90 to 100% by weight of the compound. When the liquid fraction of the pasty compound, measured at 32° C., is equal to 100%, the temperature at the end of the melting range of the pasty compound is less than or equal to 32° C.

The liquid fraction of the pasty compound, measured at 32° C., is equal to the ratio of the heat of fusion consumed at 32° C. to the heat of fusion of the pasty compound. The heat of fusion consumed at 32° C. is calculated in the same manner as the heat of fusion consumed at 23° C.

The pasty compound can be chosen from synthetic compounds and compounds of plant origin. A pasty compound may be obtained by synthesis from starting materials of plant origin.

For example, the pasty compound can be chosen from
lanolin and its derivatives,
polymeric or non-polymeric silicone compounds,
polymeric or non-polymeric fluorinated compounds,
vinyl polymers, such as, for example:
   homopolymers of olefins
   copolymers of olefins
   homopolymers and copolymers of hydrogenated dienes
   linear or branched oligomers, or homo- or copolymers of alkyl (meth)acrylates, in some embodiments having a C₈-C₃₀ alkyl group
   oligomers, homo- and copolymers of vinyl esters having C₈-C₃₀ alkyl groups
   oligomers, homo- and copolymers of vinyl ethers having C₈-C₃₀ alkyl groups,
fat-soluble polyethers resulting from polyetherification of at least one C₂-C₁₀₀, for example C₂-C₅₀, diol,
esters,
   and mixtures thereof.

In at least one embodiment, the pasty compound is polymeric, for example, hydrocarbon-based.

An example of a silicone and fluorinated pasty compound useful herein is polymethyl trifluoropropyl methylalkyl dimethylsiloxane, manufactured under the name X22-1088 by SHIN ETSU.

When the pasty compound is a silicone and/or fluorinated polymer, the composition, in at least one embodiment, comprises a compatibilizing agent, such as short-chain esters such as isodecyl neopentanoate.

Among the fat-soluble polyethers, in at least one embodiment, copolymers of ethylene oxide and/or propylene oxide with C₆-C₃₀ long-chain alkylene oxides may be used, and are chosen for example, such that the weight ratio of the ethylene oxide and/or propylene oxide to alkylene oxides in the copolymer ranges 5:95 from 70:30. In this family, there may be mentioned, for instance, copolymers such that the long-chain alkylene oxides are arranged in blocks having an average molecular weight of 1000 to 10 000, for example a polyoxyethylene/polydodecyl glycol block copolymer such as the ethers of dodecanediol (22 mol) and of polyethylene glycol (45 EO) marketed under the trade mark ELFACOS ST9 by Akzo Nobel.

Among the esters, in at least one embodiment, the following esters may be useful
esters of an oligomeric glycerol, such as diglycerol esters, for example the condensates of adipic acid and glycerol, for which a portion of the hydroxyl groups of the glycerols have reacted with a mixture of fatty acids such as stearic acid, capric acid, stearic acid and isostearic acid, and 12-hydroxystearic acid, such as, for example, those marketed under the trade mark SOFTISAN 649 by the company Sasol,
arachidyl propionate marketed under the trade mark WAXENOL 801 by Alzo,
phytosterol esters,
triglycerides of fatty acids and derivatives thereof,
pentaerythritol esters,
non-crosslinked polyesters resulting from polycondensation between a dicarboxylic acid or a linear or branched C₄-C₅₀ polycarboxylic acid and a diol or a C₂-C₅₀ polyol,
aliphatic esters of an ester resulting from the esterification of an aliphatic hydroxycarboxylic acid ester with an aliphatic carboxylic acid.

The aliphatic carboxylic acid comprises from 4 to 30, for example from 8 to 30 carbon atoms. In at least one embodiment, it is chosen from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicasanoic acid, isoarachidic acid, octyidodecanoic-acid, heneicosanoic acid, docosanoic acid, and mixtures thereof.

The aliphatic carboxylic acid, for instance, can be branched.

The aliphatic hydroxycarboxylic acid ester may be, for instance, obtained from a hydroxylated aliphatic carboxylic acid comprising from 2 to 40 carbon atoms, for example from 10 to 34 carbon atoms, such as from 12 to 28 carbon atoms, and from 1 to 20 hydroxyl groups, for instance from 1 to 10 hydroxyl groups and further, for example from 1 to 6 hydroxyl groups. The aliphatic hydroxycarboxylic acid ester is chosen from:
a) partial or total esters of saturated linear monohydroxylated aliphatic monocarboxylic acids;
b) partial or total esters of unsaturated monohydroxylated aliphatic monocarboxylic acids;
c) partial or total esters of saturated monohydroxylated aliphatic polycarboxylic acids;
d) partial or total esters of saturated polyhydroxylated aliphatic polycarboxylic acids;
e) partial or total esters of C₂ to C₁₀ aliphatic polyols which have reacted with a mono or polyhydroxylated aliphatic mono or polycarboxylic acid,
   and mixtures thereof.

The aliphatic esters of an ester are for instance chosen from:
the ester resulting from the esterification reaction of hydrogenated castor oil with isostearic acid in the 1 to 1 (1/1) ratio or monoisostearate of hydrogenated castor oil,
the ester resulting from the esterification reaction of hydrogenated castor oil with isostearic acid in the 1 to 2 (1/2) ratio or diisostearate of hydrogenated castor oil,
the ester resulting from the esterification reaction of hydrogenated castor oil with isostearic acid in the 1 to 3 (1/3) ratio or triisostearate of hydrogenated castor oil,
and mixtures thereof.

In at least one embodiment, the pasty compound is present in an amount ranging from 1 to 99%, for example from 1 to 60%, such as from 2 to 30% and further, for example, from 5 to 15% by weight of the composition.

Wax

The fatty phase of the composition according to the disclosure may comprise at least one wax. A wax, for the purposes of the present disclosure, is defined as a lipophilic fatty compound which is solid at room temperature (25° C.), with a reversible solid/liquid change of state, having a melting point greater than 40° C. and which may be as high as 200° C., and having an anisotropic crystalline organization in the solid state.

Any type of wax may be used. The waxes may be chosen from waxes of natural origin, for example of plant or animal origin, from waxes of mineral origin, from waxes of synthetic origin, and mixtures thereof. As waxes which may be used in the composition of the disclosure, there may be mentioned, for example, beeswax, Montan wax, Carnauba wax, Candelilla wax, Chinese wax, linseed wax, pine wax, cotton wax, Ouricoury wax, lignite wax, rice bran wax, sugar cane wax, Japan wax, cork fiber wax, paraffin waxes, microcrystalline waxes, lanolin wax, ozokerites, hydrogenated oils having a melting point greater than 40° C. (approximately), such as hydrogenated jojoba oil, polyethylene waxes which are derived from the polymerization of ethylene, waxes obtained by Fischer-Tropsch synthesis, fatty acid esters and glycerides which are concrete (that is to say solid) at 40° C., silicone waxes such as alkyl, alkoxy and/or esters of poly(di)methylsiloxane which are solid at 40° C., and mixtures thereof.

As indicated above, when the composition comprises a-polar oil, it comprises at least one apolar wax. When the composition comprises an apolar oil, it comprises at least one polar wax. When the composition comprises a polar oil and an apolar oil, it comprises at least one apolar wax.

As indicated above for the oils, the polarity may be described by the Hansen solubility parameter δₐ according to the equation indicated above.

The so-called apolar waxes have a δₐ value equal to 0. They are, for example, hydrocarbon waxes comprising mainly carbon and hydrogen atoms, or silicone waxes. As hydrocarbon waxes, there may be mentioned, for example, microcrystalline waxes, oxokerite, paraffin waxes, (unmodified) polyethylene waxes.

The so-called polar waxes are waxes comprising polar groups as indicated above for the oils, and they have a δₐ value greater than 0. They are for instance waxes of animal origin, waxes of plant origin, waxes of synthetic origin comprising polar groups and silicone waxes comprising polar groups. There may be mentioned, for example, Montan wax, Carnauba wax, Candelilla wax, Chinese wax, linseed wax, pine wax, cotton wax, Ouricoury wax, lignite wax, rice bran wax, sugar cane wax, Japan wax, cork fiber wax, polyglycerolated bees waxes, hydrogenated oils, fatty acid esters and glycerides which are concrete at 40° C., silicone waxes comprising at least one ester group.

The total amount of polar and/or apolar wax(es) present in the composition may range from 5 to 40% by weight, for example from 10 to 30% by weight relative to the total weight of the composition.

According to the present disclosure, the compositions disclosed herein may comprise from 0 to 10% by weight of a hydrophilic phase relative to the total weight of the composition, and further, for instance, from 1 to 5% by weight. The hydrophilic phase may comprise water and/or hydrophilic or water-soluble additives (active agents and/or gelling agents for example). In one embodiment, these additives may be chosen from moisturizers such as glycerine. The hydrophilic constituents optionally present are, for instance, dispersed in the fatty phase comprising the oils and waxes.

The composition according to the present disclosure may additionally comprise a particulate phase. The quantity of particulate phase may range from 0 to 30% by weight, such as, for example from 0 to 20% by weight relative to the total weight of the composition. When a particulate phase is present, its quantity is generally at least 0.05% by weight relative to the total weight of the composition. The quantity of particulate phase may range for example from 0.05 to 30% by weight, such as, for example from 1 to 20% by weight relative to the total weight of the composition. This particulate phase may comprise particles chosen from pigments, pearlescent agents, fillers, and mixtures thereof. These pigments, pearlescent agents and fillers are chosen from those customarily used in cosmetic compositions. As used herein, the expression “pigments” is understood to mean white or colored, inorganic or organic particles intended to color and/or opacify the composition. As used herein, the expression “fillers” is understood to mean colorless or white, inorganic or synthetic, lamellar or non-lamellar particles intended to give body or firmness to the composition, and/or smoothness, mattness and uniformity to the make-up. As used herein, the expression “pearlescent agents” is understood to mean iridescent particles which reflect light.

The pigments may be white or colored, inorganic and/or organic, of micrometer or nanometer size. There may be mentioned for example, among the inorganic pigments, titanium, zirconium or cerium dioxides, and zinc, iron or chromium oxides, and ferric blue. Among the organic pigments there may be mentioned carbon black, and barium, strontium, calcium and aluminium lacquers.

Among the pearlescent agents which can be used in the composition of the present disclosure, may be mentioned for example mica coated with titanium oxide, with iron oxide, with natural pigment or with bismuth oxychloride, and colored mica-titanium.

The fillers may be inorganic or synthetic, lamellar or non-lamellar. As fillers which may be used in the composition of the disclosure, there may be mentioned, for example, talc, mica, silica, kaolin, Nylon powders, polyethylene powders, Teflon, modified or unmodified starch, mica-titanium, natural pearl, boron nitride, microspheres such as Expancel (Nobel Industrie), Polytrap (Dow Corning), and microbeads of silicone resin (Tospearls from Toshiba, for example).

In at least one embodiment, the composition according to the present disclosure comprises at least one coloring material which may be chosen from the lipophilic colorants or hydrophilic colorants normally used in cosmetic or dermatological compositions, and from the pigments and pearlescent agents described above, and mixtures thereof. This coloring material is generally present in an amount of 0.01 to 40% by weight, and for example from 5 to 25% by weight relative to the total weight of the composition.

The composition according to the present disclosure may additionally comprise any additive customarily used in the field, for example the cosmetic field, such as antioxidants; perfumes; essential oils; preservatives; cosmetic active agents; vitamins such as vitamin E (tocopherol) and its derivatives (for example acetate), vitamin A (retinol) and its derivatives (for example retinyl palmitate), vitamin C (ascorbic acid) and its derivatives (for example ascorbyl palmitate), the derivatives of these vitamins being, for example, esters including the palmitate and acetate; essential fatty acids; sphingolipids and ceramides; self-tanning compositions such as DHA (dihydroxyacetone); sun-screening agents such as for example octylmethoxycinnamate (Parsol MCX), 3-benzophenone (Uvinul M40), butylmethoxydibenzoylmethane (Parsol 1789); surfactants; polymers; and mixtures thereof. These additives may be present in the composition in an amount of 0 to 20% by weight relative to the total weight of the composition.

Of course, persons skilled in the art will be careful to choose this or these optional additional compound(s), and/or the quantity thereof, such that the advantageous properties of the composition according to the present disclosure are not, or not substantially, impaired by the additions envisaged.

The methods for manufacturing the compositions according to the disclosure are in no way different from the methods which are conventionally used in the cosmetic field and which are entirely known to persons skilled in the art.

The compositions according to the present disclosure may, for instance, constitute a care product and/or a make-up product for keratine materials, for example the skin, the lips and the superficial body growths such as the nails, the eyelashes, the eyebrows and the hair. The make-up products are most often colored and generally comprise pigments. In the form of make-up products, the compositions of the present disclosure may, for instance, constitute a foundation, a lipstick, a blusher, an eye shadow, a mascara or an eye liner.

This product may be in the form of a cast powder, a product in a dish (foundation, blusher, eye shadow), or a product in the form of a stick (lipstick or lip care stick). In one embodiment of the present disclosure, the product is in the form of a stick, for example for lip care or for lip make-up such as lipstick.

Accordingly, the present disclosure also relates to the cosmetic use of a cosmetic composition as defined above, for the care and/or treatment of the skin, the lips and/or the superficial body growths, such as, for example for moisturizing the lips.

The present disclosure also relates to the cosmetic use of a cosmetic composition as defined above, for the making up of the skin, the lips and/or the superficial body growths.

In one embodiment of the present disclosure, a cosmetic method for the care of chapped and/or dry lips comprises applying to the lips a cosmetic composition as defined above.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific example are reported as precisely as possible. Any numerical value, however, inherently contain certain errors necessarily resulting from the standard deviation found in their respective testing measurements.

The following examples are intended to illustrate the disclosure in a non-limiting manner. The percentages are given by weight, unless otherwise stated.

### EXAMPLE 1

### Lipstick

| | |
|---|---|
| Lanolide ® (company Vevy) | 18.6% |
| Isostearyl neopentanoate | 13.3 |
| Bisglyceryl polyadipate (Softisan 100) | 3.35 |
| Phenyl trimethicone | 7.6 |
| Phenyl trimethicone 20 cSt | 3.6 |
| Bentone | 1.44 |
| Isopropyl isostearate | 22.56 |
| Ozokerite (mineral wax) | 1.75 |
| Polyethylene wax | 6.5 |
| Polyethylene wax | 3.2 |
| Titanium dioxide | 0.73 |
| Red 7 | 3.4 |
| Yellow 6 lake | 6.12 |
| Kaolin | 5 |
| N-lauroyl lysine | 2.5 |
| DL-bisabolol | 0.3 |
| BHT | qs |

Procedure:

The pigments were ground in a three roll mill with the oily phase and the gel phase. The gel was obtained with a turbine until swelling and homogenization of the bentone was obtained. The waxes were weighed in a jacketed saucepan with circulation of oil, and then heated with stirring (turbine). After complete melting, the ground material, the phenyl trimethicones and the active agents were added. After homogenization of the mixture, the juice was poured at 100° C. into a siliconized alu mould at 42° C. After recrystallization of the sticks at −4° C. for at least 30 min, they were introduced into the “ACs” and then left to stabilize for 24 h at 20° C. before any evaluation.

What is claimed is:

1. A cosmetic composition comprising, in a physiologically acceptable medium,
at least one fatty phase comprising at least one polyol ether chosen from polyalkylene glycol pentaerythritol ethers, and
at least one ester resulting from the reaction of an aliphatic alcohol with a carboxylic acid of the following formula (I): 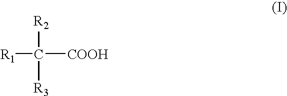
wherein R₁, R₂ and R₃, which may be identical or different, are chosen from alkyl, aryl and aralkyl radicals, and combinations thereof.

2. A composition according to claim 1, wherein R₁, R₂ and R₃, which may be identical or different, are chosen from saturated C₁-C₆ alkyl radicals.

3. A composition according to claim 1, wherein the carboxylic acid comprises a total number of carbon atoms ranging from 5 to 30.

4. A composition according to claim 1, wherein the carboxylic acid is a monoacid.

5. A composition according to claim 4, wherein the carboxylic acid is chosen from neopentanoic acid, neoheptanoic acid, neodecanoic acid, and mixtures thereof.

6. A composition according to claim 5, wherein the carboxylic acid is neopentanoic acid.

7. A composition according to claim 1, wherein the aliphatic alcohol is chosen from monoalcohols and polyols.

8. A composition according to claim 1, wherein the aliphatic alcohol comprises a number of carbon atoms ranging from 2 to 20.

9. A composition according to claim 1, wherein the aliphatic alcohol is chosen from saturated, linear or branched monoalcohols comprising from 16 to 20 carbon atoms.

10. A composition according to claim 9, wherein the at least one ester resulting from the reaction of the aliphatic alcohol with a carboxylic acid is isostearyl neopentanoate.

11. A composition according to claim 1, further comprising, in addition to the at least one alcohol ester of the acid of formula (I), at least one hydrocarbon ester.

12. A composition according to claim 11, wherein the at least one hydrocarbon ester is a branched and saturated monoester.

13. A composition according to claim 12, wherein the at least one ester and the at least one monoester are present in the composition in a ratio by mass of ranging from 2/1 to 1/5.

14. A composition according to claim 13, wherein the at least one ester and the at last one monoester are present in the composition in ratio by mass of 1/2.

15. A composition according to claim 1 1, wherein the at least one hydrocarbon ester comprises from 10 to 60 carbon atoms.

16. A composition according to claim 15, wherein the at least one hydrocarbon ester comprises from 18 to 40 carbon atoms.

17. A composition according to claim 11, wherein the at least one hydrocarbon ester is chosen from
isononanoic acid esters,
isopropyl alcohol esters,
fatty acid esters,
hydroxylated esters,
fatty alcohol heptanoates, octanoates or decanoates,
and mixtures thereof.

18. A composition according to claim 1, wherein the at least one polyol ether is chosen from polyethylene glycol pentaerythritol ethers comprising from 1 to 450 oxyethylenated units, polypropylene glycol pentaerythritol ethers comprising from 1 to 450 oxypropylenated units, and mixtures thereof.

19. A composition according to claim 18, wherein the at least one polyol ether is chosen from polyethylene glycol pentaerythritol ether comprising 5 oxyethylenated units, polypropylene glycol pentaerythritol ether comprising 5 oxypropylenated units, and mixtures thereof.

20. A composition according to claim 19, wherein the at least one polyol ether is in the form of a polyethylene glycol pentaerythritol ether comprising 5 oxyethylenated units/polypropylene glycol pentaerythritol ether comprising 5 oxypropylenated units/soya bean oil mixture.

21. A composition according to claim 1, wherein the at least one polyol ether is chosen from C₈-C₂₂ fatty alcohol ethers or mixtures of C₈-C₂₂ fatty alcohol ethers of glucose, maltose, sucrose and fructose; C₁₄-C₂₂ fatty alcohol ethers or mixtures of C₁₄-C₂₂ fatty alcohol ethers of methylglucose: and mixtures thereof.

22. A composition according to claim 1, wherein the at least one polyol ether is present in the composition in an amount ranging from 0.5 to 40% by weight relative to the total weight of the composition.

23. A composition according to claim 22, wherein the at least one polyol ether is present in the composition in an amount ranging from 1 to 30% by weight relative to the total weight of the composition.

24. A composition according to claim 1, wherein said composition is in the form of a make-up product for the body, a lipstick or gloss, a mascara, a nail varnish, a hair dyeing product, a hair care product, or a deodorant.

25. A composition according to claim 1, wherein the composition is in cast or compacted form.

26. A composition according to claim 1, wherein the composition is in the form of a lipstick.

27. A composition according to claim 1, wherein the composition comprises a particulate phase.

28. A composition according to claim 27, wherein the particulate phase comprises particles chosen from pigments, pearlescent agents, fillers, and mixtures thereof.

29. A composition comprising, in a physiologically acceptable medium,
at least one fatty phase comprising at least one polyol ether,
at least one hydrocarbon ester and
at least one non-volatile oil different from the at least one hydrocarbon ester.

30. A composition according to claim 29, wherein the at least one hydrocarbon ester comprises from 10 to 60 carbon atoms.

31. A composition according to claim 30, wherein the at least one hydrocarbon ester comprises from 18 to 40 carbon atoms.

32. A composition according to claim 29, wherein the at least one hydrocarbon ester is chosen from
isononanoic acid esters,
isopropyl alcohol esters,
fatty acid esters,
hydroxylated esters,
fatty alcohol heptanoates, octanoates or decanoates,
and mixtures thereof.

33. A composition according to claim 29, wherein the at least one polyol ether is chosen from polyethylene glycol pentaerythritol ethers comprising from 1 to 450 oxyethylenated units, polypropylene glycol pentaerythritol ethers comprising from 1 to 450 oxypropylenated units, and mixtures thereof.

34. A composition according to claim 33, wherein the at least one polyol ether is chosen from polyethylene glycol pentaerythritol ether comprising 5 oxyethylenated units, polypropylene glycol pentaerythritol ether comprising 5 oxypropylenated units, and mixtures thereof.

35. A composition according to claim 34, wherein the polyol ether is in the form of a polyethylene glycol pentaerythritol ether comprising 5 oxyethylenated units/polypropylene glycol pentaerythritol ether comprising 5 oxypropylenated units/soya bean oil mixture.

36. A composition according to claim 29, wherein the at least one polyol ether is chosen from C₈-C₂₂ fatty alcohol ethers or mixtures of C₈-C₂₂ fatty alcohol ethers of glucose, maltose, sucrose and fructose; C₁₄-C₂₂ fatty alcohol ethers or mixtures of C₁₄-C₂₂ fatty alcohol ethers of methylglucose; and mixtures thereof.

37. A composition according to claim 29, wherein the at least one polyol ether is present in the composition in an amount ranging from 0.5 to 40% by weight relative to the total weight of the composition.

38. A composition according to claim 37, wherein the at least one polyol ether is present in the composition in an amount ranging from 1 to 30% by weight relative to the total weight of the composition.

39. A composition according to claim 29, wherein said composition is in the form of a make-up product for the body, a lipstick or gloss, a mascara, a nail varnish, a hair dyeing product, a hair care product, or a deodorant.

40. A composition according to claim 29, wherein the composition is in cast or compacted form.

41. A composition according to claim 29, wherein the composition is in the form of a lipstick.

42. A composition according to claim 29, wherein the composition comprises a particulate phase.

43. A composition according to claim 42, wherein the particulate phase comprises particles chosen from pigments, pearlescent agents, fillers, and mixtures thereof.

44. A composition comprising, in a physiologically acceptable medium, at least one fatty phase comprising at least one polyol ether and at least two hydrocarbon esters.

45. A composition according to claim 44, wherein the at least two hydrocarbon esters independently comprise from 10 to 60 carbon atoms.

46. A composition according to claim 45, wherein the at least two hydrocarbon ester independently comprise from 18 to 40 carbon atoms.

47. A composition according to claim 44, wherein the at least two hydrocarbon esters are independently chosen from
isononanoic acid esters,
isopropyl alcohol esters,
fatty acid esters,
hydroxylated esters,
fatty alcohol heptanoates, octanoates or decanoates,
and mixtures thereof.

48. A composition according to claim 44, wherein the at least one polyol ether is chosen from polyethylene glycol pentaerythritol ethers comprising from 1 to 450 oxyethylenated units, polypropylene glycol pentaerythritol ethers comprising from 1 to 450 oxypropylenated units, and mixtures thereof.

49. A composition according to claim 48, wherein the at least one polyol ether is chosen from polyethylene glycol pentaerythritol ether comprising 5 oxyethylenated units, polypropylene glycol pentaerythritol ether comprising 5 oxypropylenated units, and mixtures thereof.

50. A composition according to claim 49, wherein the polyol ether is in the form of a polyethylene glycol pentaerythritol ether comprising 5 oxyethylenated units/polypropylene glycol pentaerythritol ether comprising 5 oxypropylenated units/soya bean oil mixture.

51. A composition according to claim 44, wherein the polyol ether is chosen from C₈-C₂₂ fatty alcohol ethers or mixtures of C₈-C₂₂ fatty alcohol ethers of glucose, maltose, sucrose and fructose; C₁₄-C₂₂ fatty alcohol ethers or mixtures of C₁₄-C₂₂ fatty alcohol ethers of methylglucose, and mixtures thereof.

52. A composition according to claim 44, wherein the at least one polyol ether is present in the composition in an amount ranging from 0.5 to 40% by weight relative to the total weight of the composition.

53. A composition according to claim 52, wherein the at least one polyol ether is present in the composition in an amount ranging from 1 to 30% by weight relative to the total weight of the composition.

54. A composition according to claim 44, provided in the form of a make-up product for the body, a lipstick or gloss, a mascara, a nail varnish, a hair dyeing product, a hair care product, or a deodorant.

55. A composition according to claim 44, wherein the composition is in cast or compacted form.

56. A composition according to claim 44, wherein the composition is in the form of a lipstick.

57. A composition according to claim 44, wherein the composition comprises a particulate phase.

58. A composition according to claim 57, wherein the particulate phase comprises particles chosen from pigments, pearlescent agents, fillers, and mixtures thereof.

59. A composition comprising, in a physiologically acceptable medium, at least one fatty phase comprising at least one polyol ether, at least one pasty compound other than the at least one polyol ether, and at least one hydrocarbon ester.

60. A composition according to claim 59, wherein the at least one hydrocarbon ester comprises from 10 to 60 carbon atoms.

61. A composition according to claim 60, wherein the at least one hydrocarbon ester comprises from 18 to 40 carbon atoms.

62. A composition according to claim 59, wherein the at least one hydrocarbon ester is chosen from
isononanoic acid esters,
isopropyl alcohol esters,
fatty acid esters,
hydroxylated esters,
fatty alcohol heptanoates, octanoates or decanoates,
and mixtures thereof.

63. A composition according to claim 59, wherein the at least one polyol ether is chosen from polyethylene glycol pentaerythritol ethers comprising from 1 to 450 oxyethylenated units, polypropylene glycol pentaerythritol ethers comprising from 1 to 450 oxypropylenated units, and mixtures thereof.

64. A composition according to claim 63, wherein the at least one polyol ether is chosen from polyethylene glycol pentaerythritol ether comprising 5 oxyethylenated units, polypropylene glycol pentaerythritol ether comprising 5 oxypropylenated units, and mixtures thereof.

65. A composition according to claim 64, wherein the at least one polyol ether is in the form of a polyethylene glycol pentaerythritol ether comprising 5 oxyethylenated units/polypropylene glycol pentaerythritol ether comprising 5 oxypropylenated units/soya bean oil mixture.

66. A composition according to claim 59, wherein the at least one polyol ether is chosen from C₈-C₂₂ fatty alcohol ethers or mixtures of C₈-C₂₂ fatty alcohol ethers of glucose, maltose, sucrose and fructose; C₁₄-C₂₂ fatty alcohol ethers or mixtures of C₁₄-C₂₂ fatty alcohol ethers of methylglucose; and mixtures thereof.

67. A composition according to claim 59, wherein the at least one polyol ether is present in the composition in an amount ranging from 0.5 to 40% by weight relative to the total weight of the composition.

68. A composition according to claim 67, wherein the at least one polyol ether is present in the composition in an amount ranging from 1 to 30% by weight relative to the total weight of the composition.

69. A composition according to claim 59, wherein said composition is in the form of a make-up product for the body, a lipstick or gloss, a mascara, a nail varnish, a hair dyeing product, a hair care product, or a deodorant.

70. A composition according to claim 59, wherein the composition is in cast or compacted form.

71. A composition according to claim 59, wherein the composition is in the form of a lipstick.

72. A composition according to claim 59, wherein the composition comprises a particulate phase.

73. A composition according to claim 72, wherein the particulate phase comprises particles chosen from pigments, pearlescent agents, fillers, and mixtures thereof.

74. A method for caring for and/or making up skin, lips and/or superficial body growths, said method comprising applying to said skin, lips and/or superficial body growths a cosmetic composition comprising, in a physiologically acceptable medium,
at least one fatty phase comprising at least one polyol ether chosen from polyalkylene glycol pentaerythritol ethers, and
at least one ester resulting from the reaction of an aliphatic alcohol with a carboxylic acid of the following formula (I): 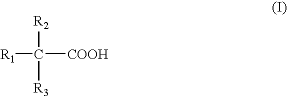
wherein R₁, R₂ and R₃, which may be identical or different, are chosen from alkyl, aryl and aralkyl radicals, and combinations thereof.

75. A method according to claim 74, wherein caring for the skin comprises moisturizing the skin

76. A method for caring for and/or making up skin, lips and/or superficial body growths, said method comprising applying to said skin, lips and/or superficial body growths a cosmetic composition comprising, in a physiologically acceptable medium,
at least one fatty phase comprising at least one polyol ether,
at least one hydrocarbon ester and
at least one non-volatile oil different from the at least one hydrocarbon ester.

77. A method according to claim 76, wherein caring for the skin comprises moisturizing the skin.

78. A method for caring for and/or making up skin, lips and/or superficial body growths, said method comprising applying to said skin, lips and/or superficial body growths a cosmetic composition comprising, in a physiologically acceptable medium, at least one fatty phase comprising at least one polyol ether and at least two hydrocarbon esters.

79. A method according to claim 78, wherein caring for the skin comprises moisturizing the skin.

80. A method for caring for and/or making up skin, lips and/or superficial body growths, said method comprising applying to said skin, lips and/or superficial body growths a cosmetic composition comprising, in a physiologically acceptable medium, at least one fatty phase comprising at least one polyol ether, at least one pasty compound other than the at least one polyol ether, and at least one hydrocarbon ester.

81. A method according to claim 80, wherein caring for the skin comprises moisturizing the skin

82. A cosmetic method for caring for chapped and/or dry lips, said method comprising applying to the lips a cosmetic composition comprising, in a physiologically acceptable medium,
at least one fatty phase comprising at least one polyol ether chosen from polyalkylene glycol pentaerythritol ethers, and
at least one ester resulting from the reaction of an aliphatic alcohol with a carboxylic acid of the following formula (I): 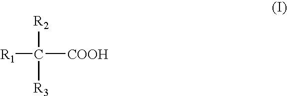
wherein R₁, R₂ and R₃, which may be identical or different, are chosen from alkyl, aryl and aralkyl radicals, and combinations thereof.

83. A cosmetic method for caring for chapped and/or dry lips, said method comprising applying to the lips a cosmetic composition comprising, in a physiologically acceptable medium,
at least one fatty phase comprising at least one polyol ether,
at least one hydrocarbon ester and
at least one non-volatile oil different from the at least one hydrocarbon ester.

84. A cosmetic method for caring for chapped and/or dry lips, said method comprising applying to the lips a cosmetic composition comprising, in a physiologically acceptable medium, at least one fatty phase comprising at least one polyol ether and at least two hydrocarbon esters.

85. A cosmetic method for caring for chapped and/or dry lips, said method comprising applying to the lips a cosmetic composition comprising, in a physiologically acceptable medium, at least one fatty phase comprising at least one polyol ether, at least one pasty compound other than the at least one polyol ether, and at least one hydrocarbon ester.
